(19) United States
(12) Patent Application Publication
(10) Pub. No.: US 2005/0015062 A1
(43) Pub. Date: Jan. 20, 2005

(54) Injection needle, method for manufacturing the injection needle and device for manufacturing the injection needle

(76) Inventor: Hisami Tamano, Yokohama-shi (JP)

Correspondence Address: KUBOVCIK & KUBOVCIK, SUITE 710, 900 17TH STREET NW, WASHINGTON, DC, 20006, US

(21) Appl. No.: 10/862,896

(22) Filed: Jun. 8, 2004

(30) Foreign Application Priority Data
Jun. 10, 2003 (JP) 2003-165495
May 25, 2004 (JP) 2004-154029

(51) Int. Cl. A61M 5/32

(52) U.S. Cl. 604/266; 156/250

(57) ABSTRACT

An injection needle comprises a base having a predetermined outer diameter and a predetermined inner diameter, a narrowed portion positioned in the front side of the base with an outer diameter and an inner diameter narrowed smaller than the outer diameter and the inner diameter of the base, respectively, and a narrowed tapered portion interposed between the base and the narrowed portion with an outer diameter gradually decreasing from the base toward the narrowed portion, wherein the narrowed portion is formed by inserting a core with an outer diameter corresponding to the inner diameter of the narrowed portion, into a metal needle pipe with an outer diameter consistent with the outer diameter of the base, to narrow the outer diameter by plastic deformation, and then by removing the core.

### BACKGROUND OF THE INVENTION

1. Field of the Invention

The present invention relates to a technique for manufacturing a minute injection needle used for hypodermic injection capable of relieving pain in puncturing skin.

2. Description of the Related Art

For description of the related art of the present invention, the applicant has made a search for patent applications and utility model registration applications to find the following technique as the related art.

The related art, JP 2002-291884 A relates to a liquid-injection needle and a liquid-injection device.

In further detail, the technique described in the document relates to a minute liquid-injection needle that is easily manufactured, that has adequate strength and low injection resistance, and is capable of relieving pain given to patients. Specifically, the liquid-injection needle includes a hollow needle for injecting liquid and a support to which the needle is secured. The needle includes a securing portion extending inside the support, and a puncture portion protruding outward from the support and capable of puncturing a living body. Then, the puncture portion has a tip with an outer diameter of 0.1 mm to 0.25 mm, and a base with an outer diameter larger than the outer diameter of the tip.

The document also describes a manufacturing method using stainless steel by, e.g. plastic processing.

### SUMMARY OF THE INVENTION

By the way, a technique for manufacturing a hollow pipe generally uses a method of rolling a flat sheet followed by welding a gap created by springback.

Since, however, the liquid-injection needle disclosed in the document has different outer diameters at its base, middle and tip, it is difficult to weld.

On the other hand, another manufacturing method has been studied of using a sheet material previously stamped off into a bottle-projected shape with minimum springback only by plastic processing without welding. The method, however, may leave a gap at a boundary and thus provide a less reliable injection needle through which liquid passes.

Moreover, for the manufacturing method only by the plastic processing, it is necessary to keep an inner diameter of a pipe from being crushed for narrowing a tip. Further, the plastic processing of rolling a flat sheet to form a pipe may cause narrowing wrinkles in an inner surface of the pipe. In some cases, the narrowing winkles are not negligible as resistance in liquid injection for a minute injection needle. Moreover, fine dust such as metal powder or oil may remain in the wrinkles.

An object of the present invention is to provide a technique for ensuring manufacture of a minute injection needle with a narrower tip.

Specifically, the present invention has the first object to keep a sufficient inner diameter in narrowing a tip in a minute injection needle with a narrower tip.

Moreover, the present invention has the second object to keep a sufficient inner diameter in narrowing a tip in a method for manufacturing a minute injection needle with a narrower tip.

Further, the present invention has the third object to smooth an inner surface of the injection needle in addition to the second object.

Moreover, the present invention has the fourth object to provide a device for manufacturing a minute injection needle with a narrower tip, capable of keeping a sufficient inner diameter in narrowing a tip.

Further, the present invention has the fifth object to smooth an inner surface of the injection needle in addition to the fourth object.

### (First Invention)

In light of the first object, the first invention of the present invention relates to an injection needle comprising a base having a predetermined outer diameter and a predetermined inner diameter, a narrowed portion positioned in the front side of the base with an outer diameter and an inner diameter narrowed smaller than the outer diameter and the inner diameter of the base, respectively, and a narrowed tapered portion interposed between the base and the narrowed portion with an outer diameter gradually decreasing from the base toward the narrowed portion, wherein the narrowed portion is formed by inserting a core with an outer diameter corresponding to the inner diameter of the narrowed portion, into a metal needle pipe with an outer diameter consistent with the outer diameter of the base, to narrow the outer diameter by plastic deformation, and then by removing the core.

In the first invention, the “front side” means a side closer to a tip when the injection needle is used for injection.

Namely, the narrowed portion of the injection needle in the invention is placed closer to the tip than the base, and the outer diameter and the inner diameter of the narrowed portion are narrowed smaller than the outer diameter and the inner diameter of the base, respectively.

Moreover, the narrowed tapered portion is interposed between the narrowed portion and the base. The outer diameter of the narrowed tapered portion decreases gradually from the base toward the narrowed portion.

The injection needle according to the present first invention is manufactured using the metal needle pipe as material. The material includes material generally suitable for an injection needle, e.g. stainless steel. Moreover, the needle pipe may be formed either by rolling a metal flat sheet followed by welding a boundary or cutting a solid cylindrical material to form an inner hole. A rear end of the needle pipe, i.e. a rear end when the injection needle is used for injection becomes the base as it is, and thus the outer diameter of the needle pipe is consistent with the outer diameter of the base.

Then, the narrowed portion is formed by inserting the core into the needle pipe to narrow the outer diameter at the tip by plastic deformation, and then by removing the core 41. The core is required to have strength to the extent that the core is not deformed by plastic deformation force of the needle pipe. Specifically, a cemented carbide wire such as a piano wire is preferable.

The outer diameter of the core corresponding to the inner diameter of the narrowed portion means the following. Specifically, releasing the plastic deformation force causes springback on the narrowed portion, and thus the inner diameter of the narrowed portion is not consistent with the outer diameter of the core but is slightly larger than that and defined by the-outer diameter of the core.

The injection needle according to the first invention has the outer diameter and the inner diameter gradually decreasing from the base, through the narrowed tapered portion, to the narrowed portion. Thus, only the narrowed portion, which is the tip to actually puncture skin, can be formed into a minute shape, and the other portions can keep larger diameters, thus preventing bending in puncture as compared with an injection needle formed minute wholly. Moreover, since the core is inserted into the inner hole in forming the narrowed portion, it keeps the inner diameter of the narrowed portion from being crushed.

In the injection needle according to the first invention, the core preferably has the outer diameter of 0.04 mm to 0.18 mm, and the narrowed portion preferably has the outer diameter of 0.1 mm to 0.25 mm and the inner diameter of 0.05 mm to 0.2 mm.

Namely, if the core has the outer diameter of 0.04 mm to 0.18 mm, the narrowed portion may have the inner diameter of 0.05 mm to 0.2 mm and thus have the outer diameter of 0.1 mm to 0.25 mm.

According to the first invention, a sufficient inner diameter can be kept in narrowing a tip in a minute injection needle with a narrower tip.

### (Second Invention)

In light of the second object, the second invention of the present invention relates to a method for manufacturing an injection needle comprising a core-insertion step on which a core is inserted into a metal needle pipe, a narrowing step on which a narrowed portion is formed by plastic deforming of a tip of the needle pipe inserted with the core to narrow its outer diameter, a core-removing step on which the core is removed, and a nib-forming step on which a tip of the narrowed portion is cut aslant to form a nib.

The material for the needle pipe is material suitable for an injection needle, e.g. stainless steel. The needle pipe maybe formed either by rolling a metal flat sheet followed by welding a boundary or cutting a solid cylindrical material to form an inner hole.

The narrowing step may include, e.g. swaging. The swaging is, while impacting the needle pipe, to diametrically compress the needle pipe successively.

In the method for manufacturing the injection needle according to the present second invention, the core is inserted into the needle pipe on the core-insertion step, then with the core being inserted, the outer diameter at the tip is narrowed on the narrowing step, and thus the inner diameter is narrowed. Further, a remaining space after the core is removed on the core-removing step keeps the inner diameter.

Furthermore, the core preferably has an outer diameter of 0.04 mm to 0.18 mm, and the narrowed portion preferably has an outer diameter of 0.1 mm to 0.25 and an inner diameter of 0.05 mm to 0.2 mm.

In order to prepare the outer diameter and the inner diameter of the narrowed area to be the size as described above, the degree of narrowing is selected appropriately.

Moreover, the needle pipe may be manufactured by a pipe-manufacturing step on which a metal flat sheet is rolled to a pipe, a welding step on which the boundary of the manufactured pipe is welded, and a cutting step on which the welded pipe is cut to a predetermined length. Thus, in the presence of the welding step, reliability for the injection needle through which liquid passes can be significantly improved as compared with the manufacturing method by plastic processing of only rolling the metal flat sheet without welding.

According to the second invention, a method for manufacturing a minute injection needle with a narrower tip, capable of keeping a sufficient inner diameter in narrowing a tip can be provided.

### (Third Invention)

In light of the third object, the third invention of the present invention further comprises, in addition to the feature of the second invention, a drilling step on which an inner hole of the narrowed portion is cut from the tip by a drill after the core-removing step.

Namely, in the presence of the drilling step, narrowing wrinkles can be cut which are created in an inner surface of the pipe by plastic processing of rolling the flat sheet to form the pipe, thus reducing surface roughness of the inner hole.

Moreover, the vicinity of the nib is thinner than other portions by the drilling step.

Furthermore, the inner hole cut by the drilling preferably has arithmetic mean roughness (Ra) of 0.05 μm to 0.2 μm and maximum height (Ry) of 0.6 μm or less. Thus, resistance in liquid injection can be reduced by smoothing the inner surface of the injection needle, and it is possible to prevent fine dust such as metal powder or oil from remaining between the narrowing wrinkles.

For the surface roughness of the inner hole within the above described range, the outer diameter and the number of revolution of the drill, and the use or kind of cutting oil, and the like are appropriately selected in connection with the material or the size of the needle pipe.

According to the third invention, the method for manufacturing an injection needle capable of smoothing an inner surface of the injection needle can be provided in addition to the advantage by the second invention.

### (Fourth Invention)

In light of the fourth object, the fourth invention of the present invention relates to a device for manufacturing an injection needle comprising a needle-securing member for securing a metal needle pipe, a core-insertion device for inserting a core into the needle pipe and removing the core from the needle pipe, and a narrowing device for narrowing apart of the needle pipe into which the-core has been inserted from its middle to its tip.

The core is, for example, a cemented carbide wire such as a piano wire.

The device for manufacturing the injection needle according to the fourth invention operates as follows.

The needle pipe may be formed either by previously rolling a metal flat sheet to form a pipe followed by welding a boundary or cutting a solid cylindrical material to form an inner hole.

The needle-securing member secures the needle pipe. Then, the core-insertion device inserts the core into the secured needle pipe. Subsequently, the narrowing device narrows a part of the needle pipe, with the core being inserted, from its middle to its tip. Since the core is inserted, the narrowing causes no blockage in the pipe hole.

Then, the core-insertion device removes the core being inserted into the needle pipe.

Thus, the injection needle having a narrower tip than its base can be manufactured.

Furthermore, the core preferably has an outer diameter of 0.04 mm to 0.18 mm.

Namely, the core is so inserted as to prevent blockage in the pipe hole even in the narrowing, but after the narrowing by the narrowing device, springback occurs on the needle pipe. Moreover, cutting by the drill is also performed after the narrowing. The above described size range is suitable for the core having an effect on these steps.

According to the fourth invention, a device for manufacturing a minute injection needle with a narrower tip, capable of keeping a sufficient inner diameter in narrowing a tip can be provided.

### (Fifth Invention)

In light of the fifth object, the fifth invention of the present invention further comprises, in addition to the feature of the fourth invention, a drill for cutting an inside of the narrowed area from the tip of the narrowed needle pipe.

The drill preferably has an outer diameter of 0.2 mm or less.

More suitable size for the outer diameter is 0.15 mm or less. Since, however, a thinner drill is more expensive and apt to be damaged, it is more preferable that the outer diameter may be 0.10 mm to 0.13 mm.

In cutting the inner hole of the needle pipe after the narrowing, the above described size range is the most suitable for easy processing or keeping arithmetic average roughness (Ra) or maximum height (Ry) within a desired range. Moreover, it is also possible to smooth the inner surface of the obtained injection needle.

According to the fifth invention, the device for manufacturing an injection needle capable of smoothing an inner surface of the injection needle in addition to the fourth invention.

### BRIEF DESCRIPTION OF THE DRAWINGS

FIG. 1 is conceptual drawing simply showing all manufacturing steps.

FIG. 2 is a sectional view showing a needle pipe.

FIG. 3 is a sectional view showing a needle-securing member and a needle pipe secured to the needle-securing member.

FIG. 4 is a sectional view showing a step of inserting a core.

FIG. 5 is a sectional view showing a state before narrowing is begun.

FIG. 6 is a sectional view showing a part of the needle pipe being narrowed.

FIG. 7 is a sectional view showing a state after the narrowing is completed.

FIG. 8 is a sectional view showing cutting by a drill.

FIG. 9 is a sectional view showing cutting by the drill.

FIG. 10 is a sectional view showing cutting by the drill.

FIG. 11 is a sectional view showing the needle pipe being removed from the needle-securing member.

### DETAILED DESCRIPTION OF THE PREFERRED EMBODIMENTS

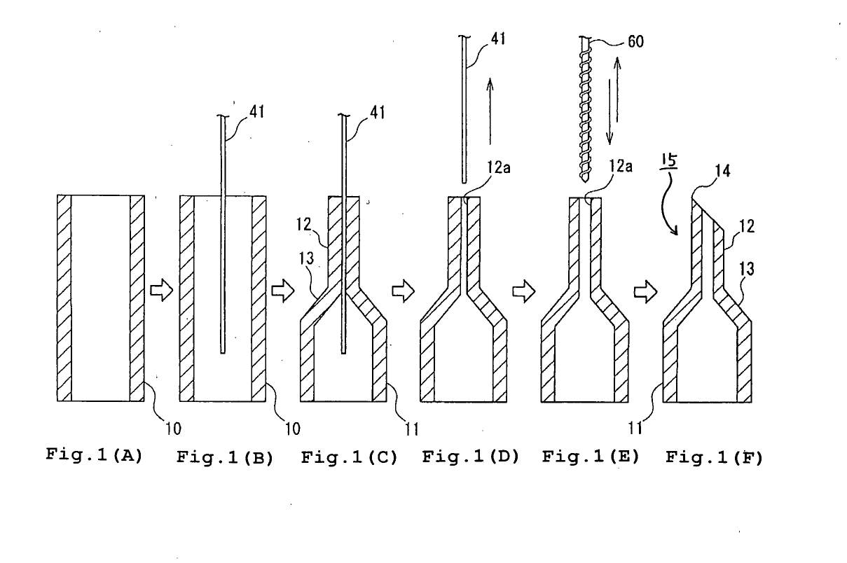

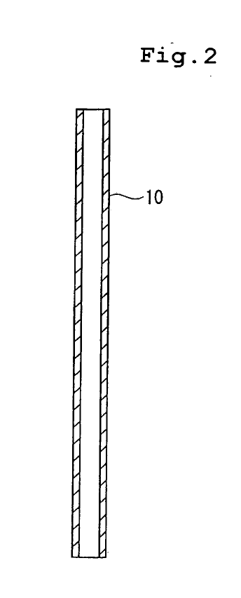

The present invention will be described hereinafter in further detail with reference to embodiments and drawings. FIGS. 1 to 11 are used herein. FIG. 1 is conceptual drawing simply showing all manufacturing steps. FIGS. 2 to 11 show a modeled device for manufacturing an injection needle according to an order of manufacturing steps.

Explanation will be made below for a device for manufacturing an injection needle that is secured to a tip of an injector for injecting liquid drug and capable of puncturing skin to inject the liquid drug from the injector into a body.

The manufacturing device comprises a needle-securing member 20 for securing a needle pipe 10 that is manufactured by rolling a stainless flat sheet to a minute pipe followed by welding its boundary and cut to a predetermined length, a needle-securing member for securing a metal needle pipe, a core-insertion device 40 for inserting a piano wire as a core 41 into the needle pipe 10 and removing the wire from the needle pipe 10, a narrowing device 30 for narrowing apart of the needle pipe 10 from its middle to its tip, and a drill 60 for cutting an inside of the narrowed area from the tip of the narrowed needle pipe 10.

The manufacturing device described above is used to manufacture an injection needle 15 via manufacturing steps shown in FIG. 1.

FIG. 1(A) shows the needle pipe 10 obtained via a pipe-manufacturing step of rolling a stainless flat sheet to manufacture a pipe, a welding step of beam-welding its boundary, and a cutting step of cutting to a predetermined length. FIG. 1(B) shows a core-insertion step of inserting a core 41 into the needle pipe 10. FIG. 1(C) shows a narrowing step of narrowing a tip with the core 41 being inserted. The narrowing is performed from an end opposite to a base 11 to the vicinity of the middle, and the narrowed area becomes a narrowed portion 12 having a smaller outer diameter than the base 11. Moreover, a narrowed tapered portion 13 having a gradually varying outer diameter is interposed between the narrowed portion 12 and the base 11.

FIG. 1(D) shows a core-removing step of removing the inserted core 41. Since springback causes an inner diameter of the narrowed portion 12 to be slightly enlarged as compared with that in the narrowing, the core 41 can easily be removed. FIG. 1(E) shows a drilling step of cutting an inner hole of the narrowed portion 12 by the drill 60. Cutting by the drill 60 causes the inner diameter of the narrowed portion 12 to be enlarged, and causes a surface of a narrow inner wall 12a to be smoother than that before cutting. FIG. 1(F) shows a nib-forming step of cutting the tip of the narrowed portion 12 aslant to form a nib 14.

FIG. 2 shows a sectional view of the needle pipe 10. Its outer diameter is 0.6 mm, its inner diameter is 0.4 mm, and its length is cut to 15 mm to 20 mm. This length is suitable for the injection needle 15 for hypodermic injection.

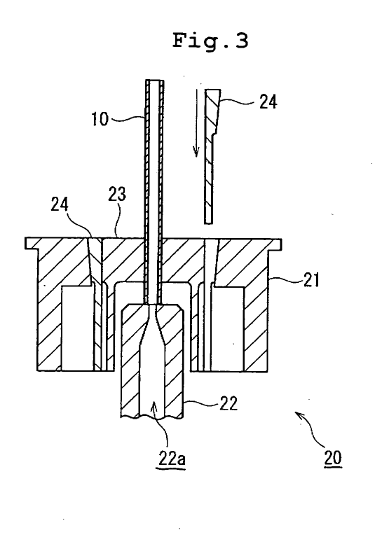

FIG. 3 shows the needle-securing member 20 and the needle pipe 10 secured to the needle-securing member 20 in the device for manufacturing the injection needle. The needle-securing member 20 comprises a support member 23 through which the needle pipe 10 penetrates to support its outer diameter direction, a resting member 22 on which a longitudinal end of the needle pipe 10 is rested for support, a main securing member 21 supported by an unshown chuck, and wedges 24 inserted between the main securing member 21 and the support member 23 to move a surface of the support member 23 contacting with the needle pipe 10 inward to secure the needle pipe 10.

The resting member 22 has a core-penetrating hole 22a in communication with an inner hole of the rested needle pipe 10. The core-penetrating hole 22a is smaller than the inner hole of the needle pipe 10 in the resting surface, but is gradually enlarged.

The core-insertion device 40 has a core 41 slightly longer than the needle pipe 10, and inserts the core 41 from the core-penetrating hole 22a of the resting member 22 in the needle-securing member 20. The core-penetrating hole 22a is gradually enlarged from the resting surface to allow easy insertion.

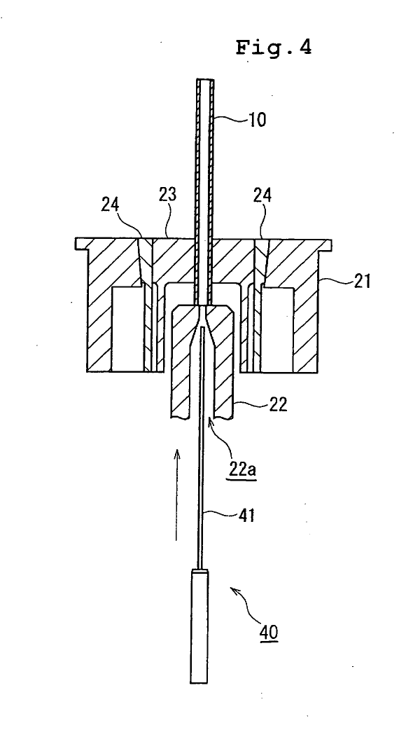

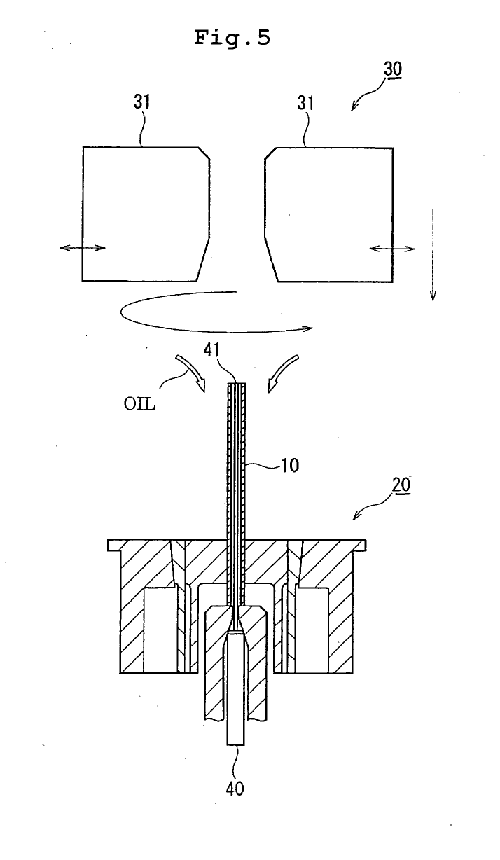

The tip of the core 41 inserted from the core-penetrating hole 22a of the resting member 22 extends from the core-penetrating hole 22a to the tip of the inner hole of the needle pipe 10 as shown in FIGS. 4 and 5. Thus, the core-insertion step is performed.

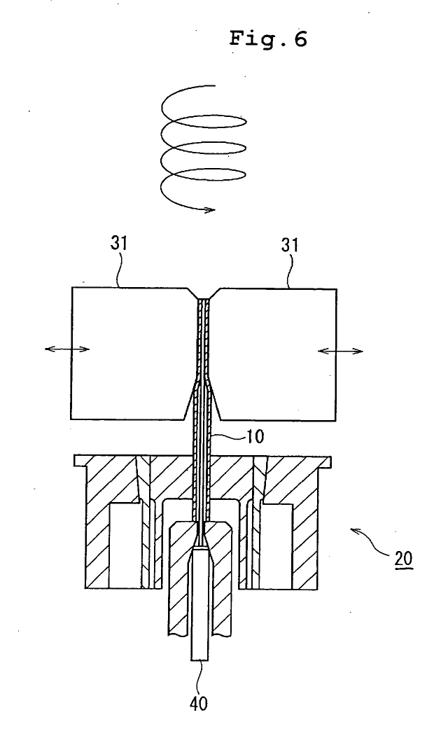

The narrowing device 30 has dies 31 holding the needle pipe 10 while rotating. The dies 31 holding the needle pipe 10 work while rotating from the tip of the needle pipe 10 as shown in FIG. 5. This working is performed by cold forging. As shown in FIG. 6, the dies 31 successively compress the needle pipe 10 while impacting the needle pipe 10 reciprocating diametrically. Thus, the narrowing step is performed. This working is referred to as swaging used for manufacturing an eyeglass frame, a steel shaft of a golf club, etc.

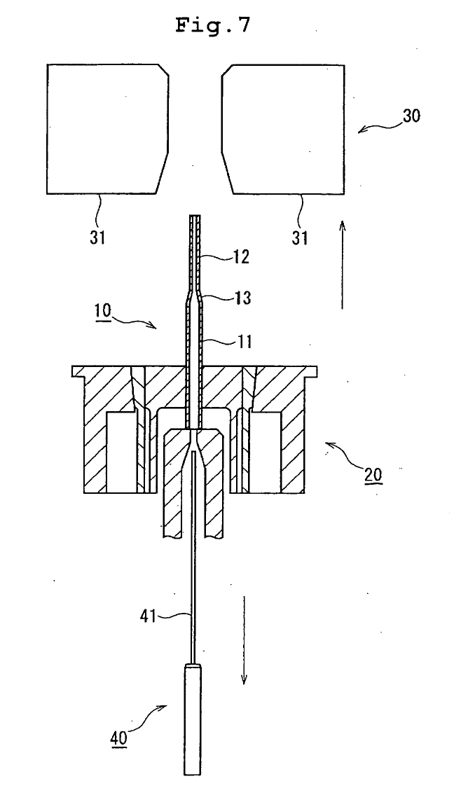

After the narrowing is performed for a predetermined length, specifically about 5 mm to 10 mm from the tip, the narrowing device 30 moves away from the needle-securing member 20 as shown in FIG. 7. Moreover, the core-insertion device 40 removes the core 41 from the needle pipe 10 as shown in FIG. 7. Thus, the core-removing step is performed. Since springback occurs on the needle pipe 10 after the narrowing device 30 moves away, the core 41 can easily be removed.

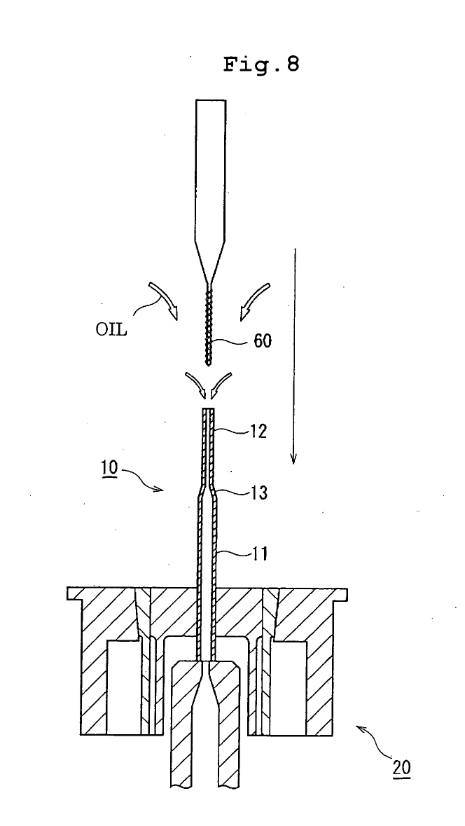

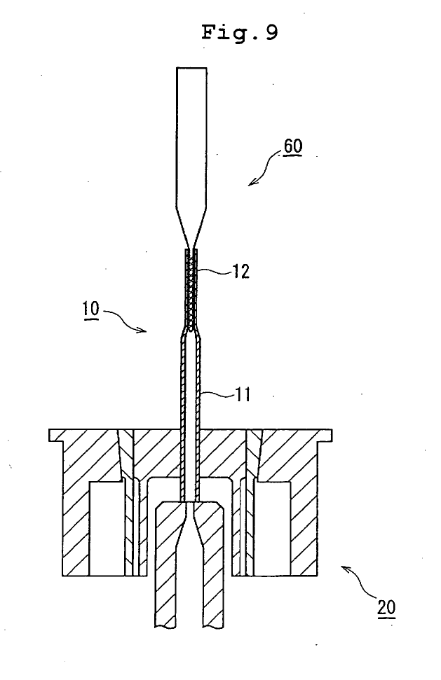

Then, as shown in FIGS. 8 and 9, the inner hole of the narrowed portion 12 is cut by the drill 60. The drill 60 has an outer diameter of 0.12 mm. The cutting is performed at 10,000 rpm with cutting oil being applied. The drill 60 has a length slightly longer than the narrowed portion 12 in the needle pipe 10, specifically, about 5 mm to 10 mm. Thus, the drilling step is performed. In this stage, the needle pipe 10 was cut along the axis, and laser measurement was performed using a non-contact three-dimensional measurement device (NH-3, Mitaka Kohki Co., Ltd.) to measure arithmetic average roughness (Ra) and maximum height (Ry) of the surface of the inner hole of the narrowed portion 12. As a result, the arithmetic average roughness (Ra) was 0.067 μm and the maximum height (Ry) was 0.315 μm.

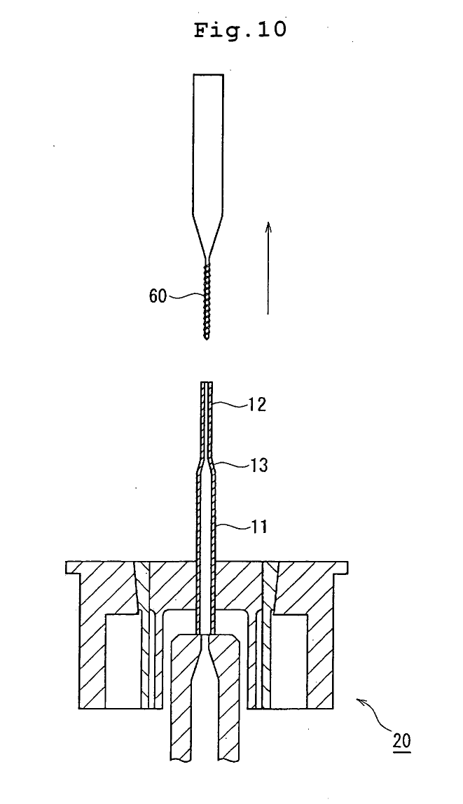

After cutting the inside of the needle pipe 10 is completed, the drill 60 is retracted as shown in FIG. 10. Then, the narrowed portion 12 thinner than the base 11 is formed. Furthermore, the cutting oil is also removed by a separate step such as a cleaning step.

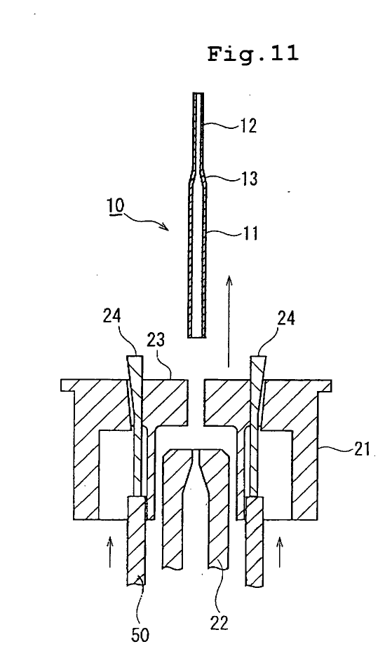

FIG. 11 shows the needle pipe 10 being removed from the needle-securing member 20. First, a disengaging device 50 impacts the support member 23 and the wedges 24, 24 to raise the wedges 24, 24, thus releasing a locked state by the wedges 24, 24. This releases force of the wedges 24, 24 holding the needle pipe 10 to disengage the needle pipe 10.

Furthermore, though detailed illustration is omitted, the needle pipe 10 manufactured as described above is formed to be the injection needle 15 by the nib-forming of cutting the drilled tip aslant to form the nib 14 (cf. FIG. 1(F)). An angle of the nib 14 is 10 to 15 degrees.

The injection needle 15 completed as described above comprises the base 11 having an outer diameter of 0.6 mm and an inner diameter of 0.4 mm, and the narrowed portion 12 having an outer diameter of 0.22 mm and an inner diameter of 0.12 mm.

### INDUSTRIAL APPLICABILITY

The injection needle according to the present invention is an injection needle for hypodermic injection used for injecting insulin or the like. However, injection needles for other uses may be manufactured by the same manufacturing method.

1. An injection needle comprising:
a base having a predetermined outer diameter and a predetermined inner diameter;
a narrowed portion positioned in the front side of the base with an outer diameter and an inner diameter narrowed smaller than the outer diameter and the inner diameter of the base, respectively; and
a narrowed tapered portion interposed between the base and the narrowed portion with an outer diameter gradually decreasing from the base toward the narrowed portion;
wherein the narrowed portion is formed by inserting a core with an outer diameter corresponding to the inner diameter of the narrowed portion, into a metal needle pipe with an outer diameter consistent with the outer diameter of the base, to narrow the outer diameter by plastic deformation, and then by removing the core.

2. The injection needle according to claim 1, wherein the core has the outer diameter of 0.04 mm to 0.18 mm, and the narrowed portion has the outer diameter of 0.1 mm to 0.25 mm and the inner diameter of 0.05 mm to 0.2 mm.

3. A method for manufacturing an injection needle comprising:
a core-insertion step on which a core is inserted into a metal needle pipe;
a narrowing step on which a narrowed portion is formed by plastic deforming of a tip of the needle pipe inserted with the core to narrow its outer diameter;
a core-removing step on which the core is removed; and
a nib-forming step on which a tip of the narrowed portion is cut aslant to form a nib.

4. The method for manufacturing an injection needle according to claim 3, wherein the core has an outer diameter of 0.04 mm to 0.18 mm, and the narrowed portion has an outer diameter of 0.1 mm to 0.25 mm and an inner diameter of 0.05 mm to 0.2 mm.

5. The method for manufacturing an injection needle according to claim 3, further comprising:
a pipe-manufacturing step on which a metal flat sheet is rolled to a pipe;
a welding step on which the boundary of the manufactured pipe is welded; and
a cutting step on which the welded pipe is cut to a predetermined length.

6. The method for manufacturing an injection needle according to claim 4, further comprising:
a pipe-manufacturing step on which a metal flat sheet is rolled to a pipe;
a welding step on which the boundary of the manufactured pipe is welded; and
a cutting step on which the welded pipe is cut to a predetermined length.

7. The method for manufacturing an injection needle according to claim 3, further comprising a drilling step on which an inner hole of the narrowed portion is cut from the tip by a drill after the core-removing step.

8. The method for manufacturing an injection needle according to claim 4, further comprising a drilling step on which an inner hole of the narrowed portion is cut from the tip by a drill after the core-removing step.

9. The method for manufacturing an injection needle according to claim 5, further comprising a drilling step on which an inner hole of the narrowed portion is cut from the tip by a drill after the core-removing step.

10. The method for manufacturing an injection needle according to claim 6, further comprising a drilling step on which an inner hole of the narrowed portion is cut from the tip by a drill after the core-removing step.

11. The method for manufacturing an injection needle according to claim 7, wherein the inner hole cut by the drilling has arithmetic mean roughness (Ra) of 0.05 μm to 0.2 μm and maximum height (Ry) of 0.6 μm or less.

12. The method for manufacturing an injection needle according to claim 8, wherein the inner hole cut by the drilling has arithmetic mean roughness (Ra) of 0.05 μm to 0.2 μm and maximum height (Ry) of 0.6 μm or less.

13. The method for manufacturing an injection needle according to claim 9, wherein the inner hole cut by the drilling has arithmetic mean roughness (Ra) of 0.05 μm to 0.2 μm and maximum height (Ry) of 0.6 μm or less.

14. The method for manufacturing an injection needle according to claim 10, wherein the inner hole cut by the drilling has arithmetic mean roughness (Ra) of 0.05 μm to 0.2 μm and maximum height (Ry) of 0.6 μm or less.

15. A device for manufacturing an injection needle comprising:
a needle-securing member for securing a metal needle pipe;
a core-insertion device for inserting a core into the needle pipe and removing the core from the needle pipe; and
a narrowing device for narrowing a part of the needle pipe into which the core has been inserted from its middle to its tip.

16. The device for manufacturing an injection needle according to claim 15, wherein the core has an outer diameter of 0.04 mm to 0.18 mm.

17. The device for manufacturing an injection needle according to claim 15, further comprising a drill for cutting an inside of the narrowed portion from the tip of the narrowed needle pipe.

18. The device for manufacturing an injection needle according to claim 16, further comprising a drill for cutting an inside of the narrowed portion from the tip of the narrowed needle pipe.

19. The device for manufacturing an injection needle according to claim 17, wherein the drill has an outer diameter of 0.2 mm or less.

20. The device for manufacturing an injection needle according to claim 18, wherein the drill has an outer diameter of 0.2 mm or less.
